(19) United States
(12) Patent Application Publication
(10) Pub. No.: US 2025/0216357 A1
(43) Pub. Date: Jul. 3, 2025

(54) HIGH-TEMPERATURE REFERENCE ELECTRODE, VENTURI FLOW TIP FOR A HIGH-TEMPERATURE REFERENCE ELECTRODE, AND ENGINEERED SALT BRIDGE FOR A HIGH-TEMPERATURE REFERENCE ELECTRODE

(71) Applicant: HiFunda, LLC, Salt Lake City, UT (US)

(72) Inventors: James J. Steppan, Park City, UT (US); Byron S. Millet, Woods Cross, UT (US); Lee C. Sorensen, West Jordan, UT (US); Thomas A. Meaders, West Valley City, UT (US)

(21) Appl. No.: 19/004,055

(22) Filed: Dec. 27, 2024

Related U.S. Application Data
(60) Provisional application No. 63/615,935, filed on Dec. 29, 2023.
(60) Provisional application No. 63/615,940, filed on Dec. 29, 2023.
(60) Provisional application No. 63/615,948, filed on Dec. 29, 2023.

(51) Int. Cl. G01N 27/30; G01N 27/40

(52) U.S. Cl. CPC G01N 27/301; G01N 27/40

(57) ABSTRACT

A high-temperature reference electrode (HTRE) for measuring electrochemical characteristics of a molten salt test composition, includes a metal housing tube which defines an inner lumen, a thermocouple reference wire disposed within the metal housing tube inner lumen and spaced apart from the inner surface, a molten reference salt disposed in the metal housing tube in contact with the thermocouple reference wire, and a porous membrane disposed at a bottom end of the metal housing tube. The thermocouple includes a reference metal. The molten reference salt includes a salt corresponding to the molten salt test composition and a salt of the reference metal. A venturi flow tip or end cap for a HTRE may be used with flowing high-temperature molten salts. An engineered salt bridge may be used to fabricate a HTRE using a commercial off-the-shelf low-temperature reference electrode.

### CROSS-REFERENCE TO RELATED APPLICATIONS

This application claims the benefit of U.S. Provisional Application No. 63/615,935, filed Dec. 29, 2023, U.S. Provisional Application No. 63/615,940, filed Dec. 29, 2023, and U.S. Provisional Application No. 63/615,948, filed Dec. 29, 2023, which applications are incorporated herein by reference.

### STATEMENT REGARDING FEDERALLY SPONSORED RESEARCH OR DEVELOPMENT

This invention was made with government support under Award Number: DE-SC0020579 awarded by the U.S. Department of Energy and titled, STABLE HIGH-TEMPERATURE MOLTEN SALT REFERENCE ELECTRODES. The government has certain rights in the invention.

### BACKGROUND

The disclosed invention relates to thermodynamic, high-temperature reference electrodes (HTREs) suitable for use with high-temperature molten salts. As used herein, the “high-temperature” reference electrodes may operate at a temperature in the range of 500° C. to 1000° C.

Molten salts are used in various industrial applications. In one application, molten salts are used as a heat transfer fluid. Metal pipes and fittings used in various industrial applications can be susceptible to deterioration over time, such as due to corrosion, when pipes are carrying molten salts. This requires that pipes be monitored to assess the rates of deterioration and to attend to scheduled maintenance for replacement of damaged pipes. The corrosiveness of molten salts may be determined by monitoring the open circuit potential (OCP) of the molten salt during operation. This is accomplished by immersing a reference electrode in the molten salt to enable real-time monitoring of the OCP (also referred to as the reduction/oxidation potential, or redox potential) of the molten salt. Chemical changes in the molten salt can be detected to prevent corrosion of reactor components.

Developing next-generation nuclear power generation will have a significant positive effect on power generation and CO₂ emissions in the United States and worldwide. Such development requires improved thermodynamic high-temperature reference electrodes for controlling molten salt chemistry and monitoring corrosion of structural materials in molten salt-cooled reactors and fuel processing equipment.

Thermodynamic, high-temperature reference electrodes (HTREs) with lifetimes of up to 6 months are important for monitoring the redox potential of nuclear-relevant, molten salts at elevated temperatures for corrosion control. Zhang, J. Forsberg, C. Simpsons, M. et al. “Redox potential control in molten salt systems for corrosion mitigation.” Corrosion Science 144 (2018): 44-53. Unfortunately, robust thermodynamic HTREs for this challenging application are not commercially available, and HTREs that have been evaluated are custom designs fabricated by researchers that last on the order of days. Researchers typically make their own HTREs with inherent variability due to design (reference wire, membrane type, seals, etc.) and fabrication methods and personnel (skilled technician vs. graduate student, etc.) differences. The lack of commercially available standardized HTREs drives hidden factory costs (time, expenditures) as researchers are expending time and effort making HTREs instead of focusing on electrochemical R&D and process/product development. HTRE designs built by researchers are often not capable of being packaged and shipped to different locations.

There is a need in the art for robust thermodynamic HTREs which are standardized and customized.

The disclosed invention further relates to a venturi flow tip or end cap for a high-temperature reference electrodes (HTRE) suitable for use with flowing high-temperature molten salts.

The HTRE technology disclosed herein was developed and tested primarily in high-temperature molten salts (HTMS) within crucibles where the HTMS is stagnant. When a high-temperature reference electrode is inserted into a flowing molten salt, such as in a flow-loop, the upstream side of the HTRE tip and membrane are vulnerable to a higher rate of erosion and corrosion. It would be an advancement to provide an end cap to reduce or limit erosion and corrosion of the HTRE tip and membrane.

The flow across the membrane can also enhance the wicking and diffusion of the reference high-temperature molten salt through the membrane. It would be an advancement to reduce or limit wicking and diffusion of the reference high-temperature molten salt through the membrane.

The disclosed invention further relates to an engineered salt bridge for a high-temperature reference electrode.

The disclosed invention relates to an engineered salt bridge which enables the use of a commercial off-the-shelf (COTS) reference electrode, operating at near-ambient temperature (30 to 50° C.), to measure conditions of a high-temperature molten salt system at a temperature range from about 500 to 1000° C.

Many commercial off-the-shelf (COTS) reference electrodes (REs) are readily available from suppliers such as BAS, Pine, Gamry, Koslow, and Ametek, in a number of different chemistries and configurations, such as Ag/AgCl, saturated calomel electrode (SCE), mercury/mercurous-sulfate, Ni/NiF₂, Ag/AgF₂, etc. COTS REs are designed to function well at operating temperatures in the range of 30 to 50° C. COTS REs generally not suitable for use with high-temperature molten salt systems which operate at a temperature range from about 500 to 1000° C.

It would be an advancement in the art to use a relatively low cost, commercially available reference electrode in high-temperature molten salt systems.

### SUMMARY OF THE INVENTION

The disclosed invention relates to thermodynamic, high-temperature reference electrodes (HTREs) suitable for use with high-temperature molten salts. The disclosed invention further relates to a venturi flow tip or end cap for a high-temperature reference electrodes (HTRE) suitable for use with flowing high-temperature molten salts. The disclosed invention further relates to an engineered salt bridge permitting the use of a commercial off-the-shelf low-temperature reference electrode for a high-temperature reference electrode.

One general aspect includes a high-temperature reference electrode for measuring electrochemical characteristics of a molten salt test composition. The high-temperature reference electrode includes a metal housing tube having an inner surface which defines an inner lumen. The electrode also includes a thermocouple reference wire disposed within the metal housing tube inner lumen and spaced apart from the inner surface. Thermocouple reference wire may comprise a reference metal. The electrode also includes a molten reference salt disposed in the metal housing tube in contact with the thermocouple reference wire. The molten reference salt may include a salt corresponding to the molten salt test composition and a salt of the reference metal. The electrode also includes a porous membrane having pores. The porous membrane may be disposed at a bottom end of the metal housing tube.

Implementations may include one or more of the following features. The metal housing tube of the high-temperature reference electrode may include an inner electrical insulating coating at the inner surface. The inner electrical insulating coating may include an insulating material selected from non-conductive diamond and boron-nitride.

The high-temperature reference electrode may include one or more electrically insulating spacers disposed within the metal housing tube to retain the thermocouple reference wire within the metal housing tube.

The metal housing tube may include an outer surface and an outer electrical insulating coating at the outer surface. The outer electrical coating may include an insulating material selected from non-conductive undoped diamond, boron nitride, and silicon carbide.

The porous membrane may include a plurality of pores. The porous membrane may include two or more plates, where each plate may include a plurality of aligned pores and where the plates are offset to cause the pores from one plate to partially overlap the pores of an adjacent plate. The porous membrane may include a porous membrane plate and a membrane support which has a thickness greater than the porous membrane plate. The porous membrane may include a substrate a plurality of pores formed in the substrate, where the substrate has a reduced thickness at location where the pores are formed. The porous membrane may include a material chemically compatible with the molten salt test composition selected from mullite, magnesia (MgO), graphite, porous stainless steel, and ion conducting membranes.

The reference metal may include Ag. The reference metal may include Ni.

The metal housing tube may comprise a metal which is chemically compatible with the molten salt test composition and the molten reference salt. The metal housing tube may comprise a metal selected from Ag, Ni, 316-stainless steel, and a nickel-chromium-based superalloy.

The metal housing tube has an inner surface which may include an electrical insulating coating.

The high-temperature reference electrode may comprise a stopper seal disposed at a top end of the metal housing tube. The stopper seal may include a center hole to allow the thermocouple reference wire to pass therethrough.

The high-temperature reference electrode may include a housing lead wire electrically connected to an exterior surface of the metal housing tube and a reference lead wire electrically connected to the thermocouple reference wire.

The high-temperature reference electrode may include an electrode wire connected to an outside surface of the metal housing tube and electrically isolated from the outside surface of the metal housing tube.

One aspect includes a venturi flow tip designed to be placed over any type of reference electrode designed for use within a flowing molten salt. The reference electrode may include a porous membrane or frit, a housing, and a reference wire. The flow tip may be screwed on or permanently attached via welding, brazing, or other chemically and thermally compatible method. The flow tip may be removable and replaceable. The venturi flow tip uses a venturi effect to draw small samples of the molten test salt into the flow tip's internal chamber for communication with the HTRE's reference salt through a porous membrane. The flow tip has an upstream shield with a hole or series of holes at the leading edge of the flow where a stagnation occurs, creating a stagnation (high) pressure. The downstream end of the flow tip has a small opening or series of openings where the external flow of the test salt around the cap creates a static (low) pressure.

The difference in pressure between the stagnation pressure and the static pressure creates a flow of the test salt into the flow tip chamber. The size and/or location of the entrance and exit holes and the shape of the venturi tip may be varied to provide a desired pressure difference.

The flow tip may contain a baffle just below the porous membrane to further shield the HTRE's membrane from the pressure effects and flow effects within the flow tip. In this embodiment the entrained test salt diffuses through the macro-openings of the baffle and then through the micro-openings in the porous membrane.

The venturi tip may be adapted for use in a three-electrode HTRE design. The working electrode wire may be inserted into the chamber within the end cap. The working electrode is electrically insulated from the cap and HTRE housing using an insulation bead or other method such as coating the wire. Any electrically insulating method and material that is chemically and thermally compatible may be used, such as beads or wire coatings of boron-nitride, nonconductive diamond, or other similar material.

Another aspect includes an engineered salt bridge to provide a means of utilizing commercial off-the-shelf (COTS) reference electrodes (RE) in high-temperature (500 to 1000° C.) molten salts. The engineered salt bridge uses a salt bridge or a Luggin capillary or extension tube and frit that enables ionic communication with the room-temperature COTS RE and the high-temperature test melt and prevents contamination of the reference melt, gel, or solution with the test melt and vice-versa. The reference melt may be any type of molten salt including MgCl₂, KCl, NaCl, AgCl, FLiNaK, FLiBe, or other salt of interest.

A controlled porosity frit or membrane is sealed to the hot or distal end of the extension tube. The thermal gradient in the extension tube is controlled by design to keep the cold or proximal end of the COTS RE at room temperature so that COTS REs that are not designed for high-temperature can be used.

The walls of the RE extension tube may be thinned in areas to minimize heat transfer from the high-temperature melt to the COTS RE. Cooling fins may also be attached to the extension tube to promote heat transfer away from the tube.

The COTS RE is placed through a seal in the upper end of the RE extension tube. The liquid reference melt within the lower extension tube is molten where the RE extension tube is submerged in the test melt and transitions to a solid in the zone outside the test melt where the temperature has cooled to less than the reference salt melting point. The solid portion of the reference salt creates a passive seal the prevents contamination both ways between the COTS RE reference salt and the test melt.

Above the solid reference melt is a bridge salt. Nonlimiting examples of the bridge salt include a conductive gel such as KCl dissolved in agar or a gel comprising KCl, glycerol, and cellulose gum. The COTS RE is inserted into the bridge salt and communicates through the frit or porous membrane of the COTS RE.

An optional secondary membrane and compartment may be used between the solid reference melt and conductive gel to minimize contamination or to have a graded concentration. The salt bridge extension is not limited to a straight linear design and may be any shape to accommodate deployment. A nonlimiting example of an alternative shape is an S shape.

### DESCRIPTION OF THE DRAWINGS

FIGS. 1A to 1C disclose the basic components of the high-temperature reference electrode.

FIGS. 2A and 2B disclose a porous membrane with a generally thick substrate and localized areas machined to a thickness suitable for laser-drilled pores.

FIGS. 3A to 3D show multiple thin plates with laser-drilled pores stacked to provide more robustness and to allow for a smaller pore size by stacking slightly offset pores.

FIGS. 4A and 4B show a membrane support component with holes to allow molten salt transfer to the porous membrane and to provide structural support.

FIGS. 5A to 5C show a design kit that permits users to assemble HTRE components after the users fill the housing with their customized reference salt.

FIGS. 6A and 6B show a HTRE having a third electrode connected to the outside of the housing tube which can function as a working electrode, enabling the HTRE to be used as a 3-electrode electrochemical cell.

FIGS. 7A to 7D show a venturi flow tip designed to be placed over any type of HTRE.

FIGS. 8A and 8B show an example of the venturi flow tip in a three-electrode HTRE design.

FIGS. 9A and 9B show a representation of an engineered salt bridge for use with commercial off-the-shelf reference electrodes in high-temperature (500 to 1000° C.) molten salts.

FIGS. 10A and 10B show the salt bridge design incorporated into a high-temperature reference electrode (HTRE).

### DESCRIPTION OF THE INVENTION

### High-Temperature Reference Electrode

The disclosed invention relates to a standardized and customized high-temperature reference electrode (HTRE). The HTRE may include integrated smart logic and digital output that will reduce variability in collaborative electrochemical testing for a wide range of high-temperature molten salt (HTMS) applications.

The following disclosure includes details of the design, fabrication and assembly, and calibration of the HTRE described herein.

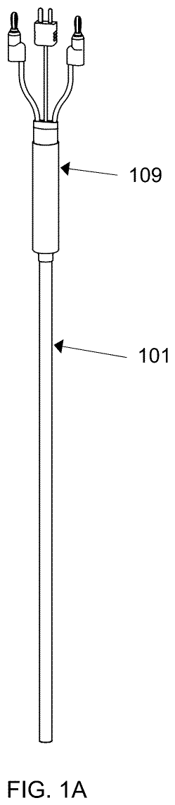

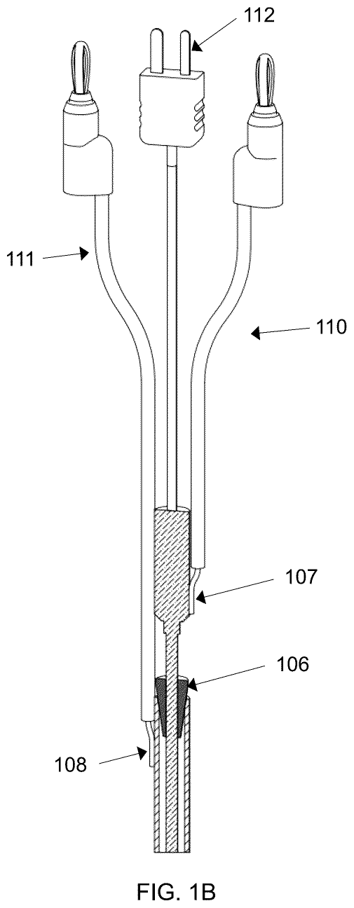

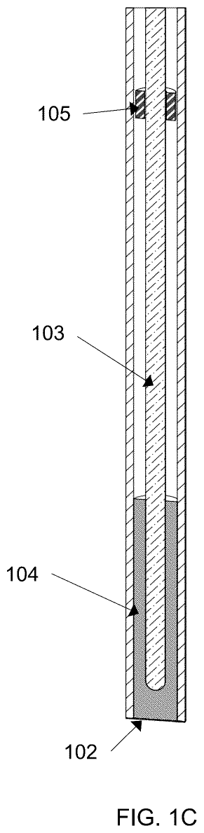

FIGS. 1A to 1C discloses the basic components of the HTRE, which consist of the following:

### 1. Porous Membrane.

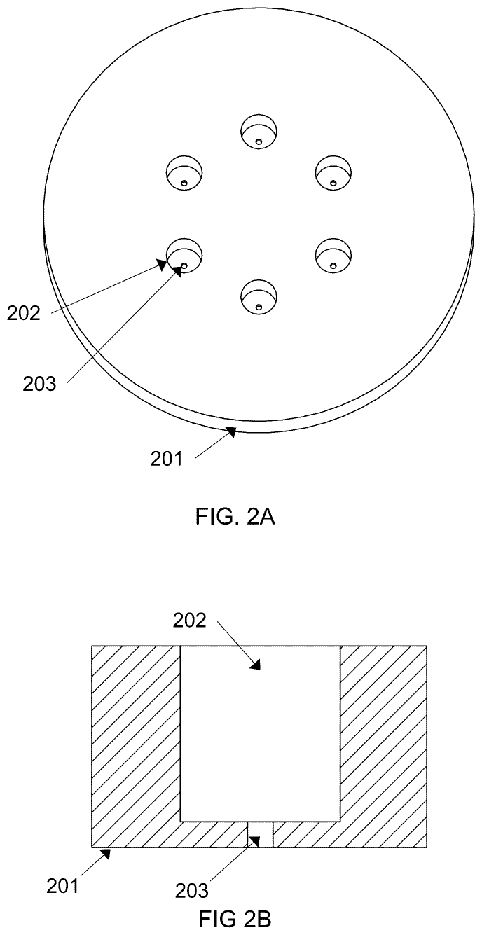

The porous membrane (102), shown in FIG. 1C, contains sub-micron to micron sized pores that are sized for tailored leak rates. The pores may be produced via subtractive processing such as laser drilling or chemically etching or via additive manufacturing or hybrid approaches such as electroforming over a mold that is subsequently chemically removed or melted. In an embodiment, the pores may be laser-drilled, which requires a very thin membrane of ˜0.005″, which is not structurally robust and can rupture or crack. Therefore, a thicker substrate shown in FIGS. 2A to 2B may be used to provide better structural support between the pores (203). The thicker substrate (201) may be machined to remove substrate material from region (202) to a thickness only at the locations where the pores (203) will be laser drilled, as shown best in FIG. 2B.

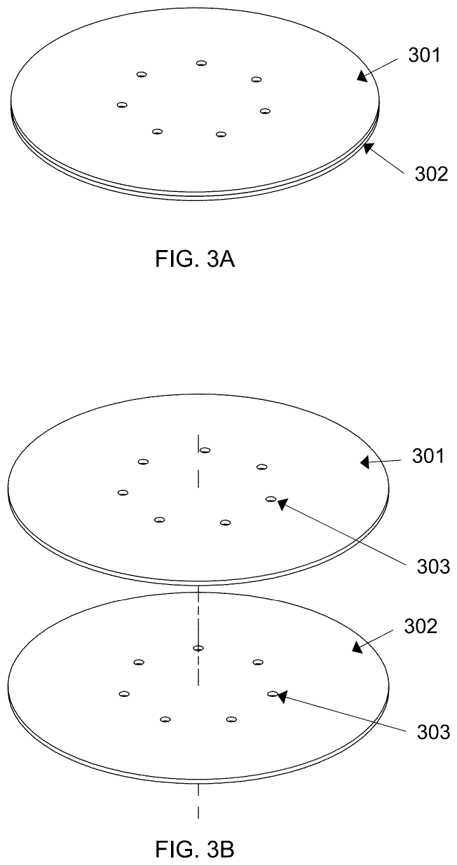

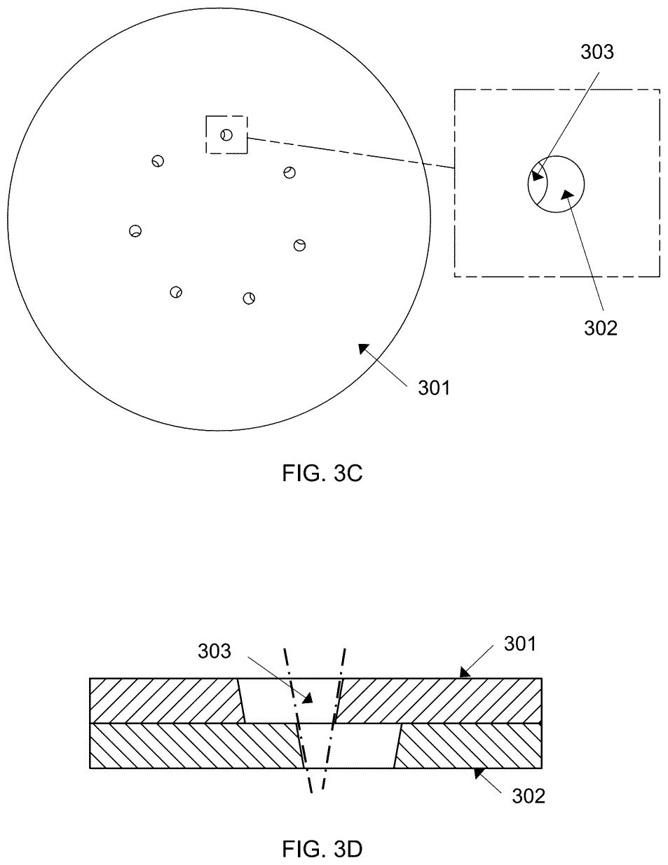

Alternatively, FIGS. 3A to 3D show multiple thin plates (301 and 302) with laser-drilled pores (303) may be stacked to provide more robustness and also allow for a smaller pore size by stacking slightly offset pores over each other, resulting in a smaller opening where the pores overlap, as shown in FIGS. 3C and 3D.

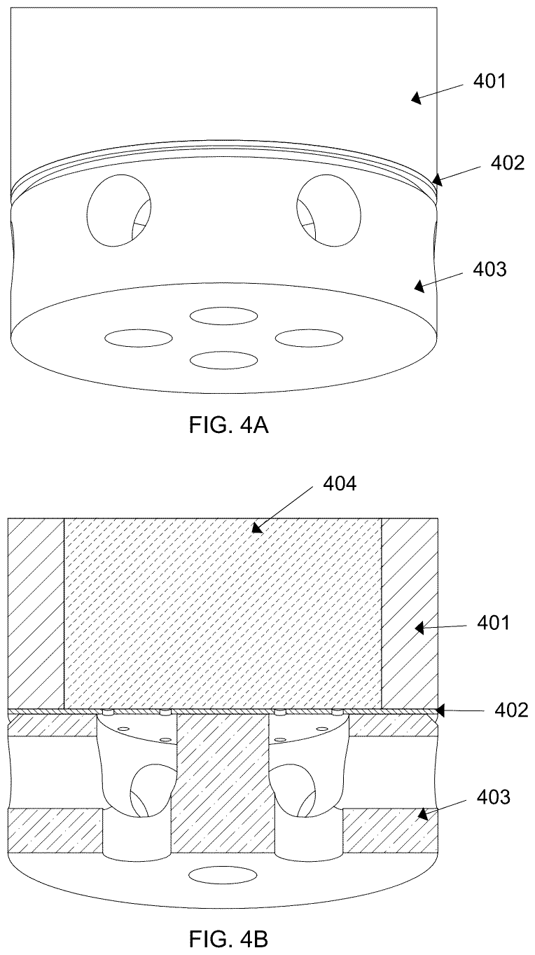

A membrane support component, with holes to allow for molten salt transfer to the porous membrane may also be used as a method to supply structural support, as shown in FIGS. 4A and 4B. In this configuration a thin membrane (402) is welded to the housing tube (401) to contain the molten reference salt (404). The membrane support (403) is welded to the membrane so that the membrane cannot flex and burst.

Other membrane options include mullite, magnesia (MgO), graphite, porous stainless steel, and other materials that are chemically compatible with the HTMS and may include ion-conducting membranes.

### 2. Molten Reference Salt

The high-temperature molten salt (HTMS) (see 104 in FIG. 1C) chemistry for the reference compartment may vary depending on the bulk salt chemistry in the system that the electrode will be monitoring. The reference melt is of the same composition as the test melt plus the salt of the reference metal forming the reference wire. For example, if the reference wire is Ag, then the salt of the reference metal would be a salt comprising Ag+, such as AgCl. Nonlimiting examples include Ag/AgCl+mixed chlorides for testing in mixed chlorides, Ni/NiF₂/FLiNaK for testing in FLiNaK, and Ni/NiF₂/FLiBe for testing in FLiBe. Mixed chlorides include one or more of the following NaCl, KCl, CsCl, CaCl₂, MgCl₂, and BaCl₂.

### 3. Metal Housing Tube

The housing of the HTRE (See 101 in FIG. 1A) is a metal tube for robustness and electrical conductivity, and may include any metal that is chemically compatible with the salt chemistry in both the HTRE reference salt and the bulk salt at the required temperature (typically greater than 550° C.). Nonlimiting examples include Ag, Ni, 316-Stainless Steel, and Inconel. Ni and 316-Stainless Steel have the advantage that they are less thermally conductive and will not transfer as much heat from the bulk salt, which will also result in less temperature increase of the HTRE.

The tube may be electrically insulated on the inside to prevent electrical conductivity between the housing and the thermocouple reference wire, allowing for the outer housing diameter to be utilized for open circuit potential (OCP) measurements that do not have an offset caused by electrical contact with the reference melt and inside of the housing. The electrical insulating coating needs to be chemically and thermally compatible with the reference salt and use-temperature. The insulating coating may utilize materials such as non-conductive diamond or boron-nitride.

If the inner diameter of the tube is not electrically insulated, then insulating beads (105) will be required to separate the thermocouple reference wire (103) from the inner diameter of the tube. These beads must be able to withstand the use-temperature, and ideally would be chemically compatible with the reference salt. Non-limiting examples of materials that may be used for the insulating beads include steatite and boron-nitride.

### 4. Thermocouple Reference Wire

The thermocouple sheath functions as the thermocouple reference wire in the HTRE as shown by (103) in FIGS. 1C. The thermocouple has two purposes; 1) its sheath is a reference wire for measuring the reference voltage within the reference molten salt, and 2) the electrically isolated thermocouple measures the temperature of the bulk salt via a thermocouple connector (112). To act as a suitable reference wire, the sheath of the thermocouple must be a material that is chemically compatible with the salt at the required test temperature. Also, the reference wire should reach an equilibrium potential for the HTMS system being measured. Non-limiting examples of the sheath material include Ag or Ni. Making an Ag thermocouple sheath may be difficult due to the low melting temperature of Ag; therefore, a separate Ag tube is placed over a thermocouple sheath of any other metal and welded to the base of the thermocouple to create a seal.

### 5. Stopper Seal

A stopper (106) seals the top of the housing tube and around the thermocouple as shown in FIG. 1B. A hole must be present through the middle of the stopper to allow the thermocouple to penetrate through. The stopper material must be able to withstand the temperature of the HTRE at the top of the housing tube, which may be significantly lower than the bulk salt temperature due to cooling along the length of the tube. Silicone or other high-temperature elastomers would be a good material for the stopper. Since the stopper may be exposed to fumes from the reference salt, chemical compatibility of the stopper and reference salt fumes should be considered. A high-temperature epoxy may be used to secure the stopper in place and provide a secondary seal.

### 6. Electrical Lead Wires

Wires attached to the housing tube (111) and the thermocouple sheath (110), as shown in FIG. 1B, may be used to provide electrical connections for performing electrical measurements of the HTRE as shown in FIG. 1B. The lead wires may be welded or brazed to the housing at location (108) and thermocouple sheath at location (107) to provide a secure high-temperature connection. The lead wires provide the advantage of being able to move the electrical connections to equipment away from the high-temperatures of the HTRE without having to use alligator clips or other method of attachment that may not be reliable at high-temperatures.

### 7. Handle

The top-end of the HTRE may have a handle (109), as shown in FIG. 1A. The handle may be custom molded onto the housing tube using silicone or epoxy, or it may be an off-the-shelf or custom molded boot that is slid over the housing. The handle will provide the end user with a convenient structure for handling the HTRE. The handle may also serve as a cosmetic feature to cover wire connections and seals.

### Other Novel Design Features Include:

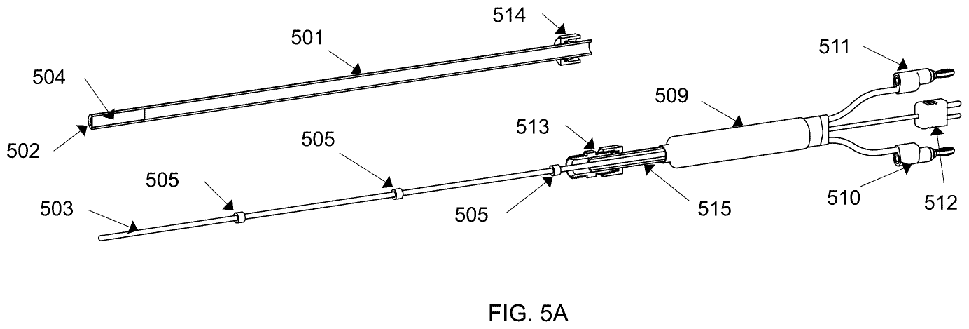

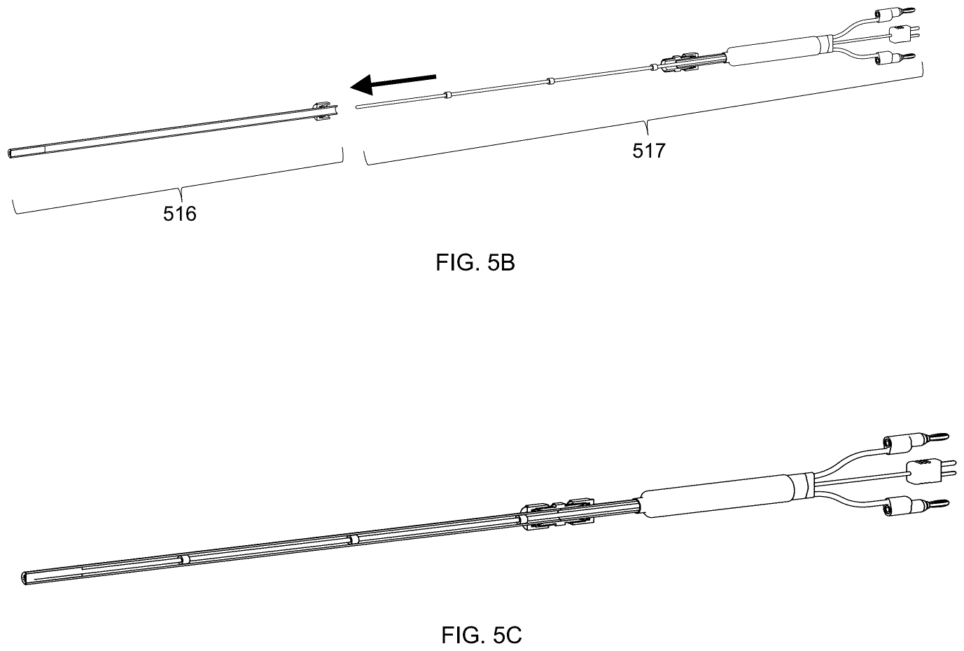

The HTRE disclosed herein may include a design kit that provides users with HTRE components that can be easily assembled after the user fills the housing with their customized reference salt, as shown in FIG. 5A through 5C. This design kit enables end users the ability to charge and seal the reference compartment of the HTRE, defined by the housing tube (501) and the porous membrane (502), with nuclear-relevant salts (uranium, thorium, and others) of their interest. The kit includes a prebuilt bottom-end (516) and top-end (517). The assembly for the end user would involve filling the salt (504) into the bottom-end housing (501) that already has the porous membrane (502) and housing lead wire (511) attached. Inserting the top-end that includes the thermocouple (503) that is sealed to a tube (515) that mates to the housing tube (501) of the bottom-end, the thermocouple lead wire (512) and the reference lead wire (510), insulating beads (505), and a handle (509). The end user would then seal the mating tube on the top-end to the housing tube on the bottom-end. Non-limiting examples of sealing methods include a compression nut and ferrule (514) that is tightened onto a pre-swaged or welded fitting (513), a gasket or O-ring that is compressed using a threaded connection, epoxy, silicone, or other similar sealing method.

The HTRE may contain electronics that incorporate an analog to digital converter in the top-end of the HTRE or within the handle. This is advantageous in that the digital signal can be transported through signal wires with less susceptibility to noise than an analog signal and minimizes the number of feedthroughs for the test glovebox. It also allows for direct digital processing of the signal within the HTRE, as noted in the following design features.

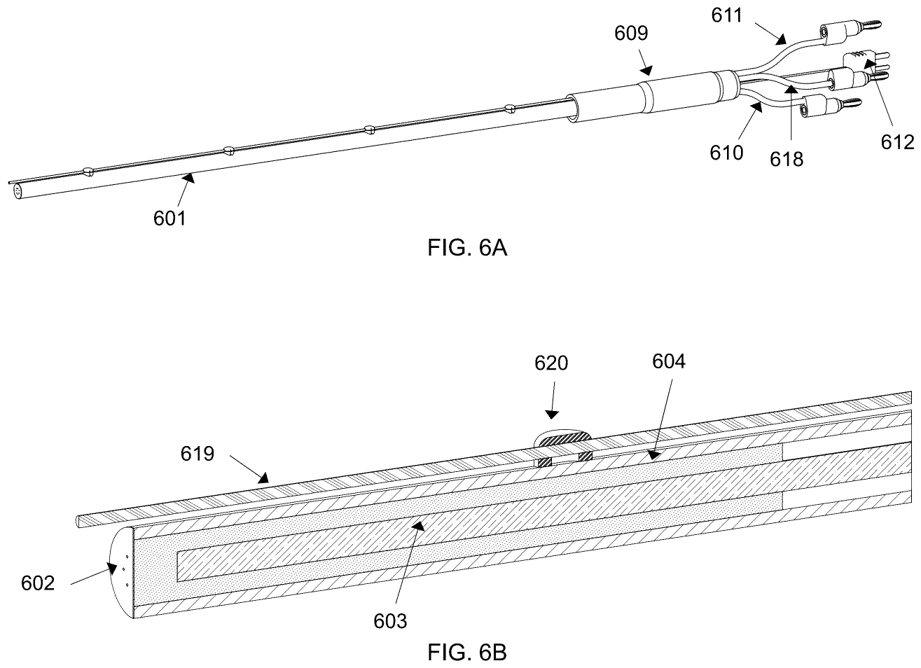

A third electrode wire may also be included in the HTRE as shown in FIG. 6A and FIG. 6B. The HTRE shown in FIGS. 6A and 6B is similar to the HTRE disclosed herein with a housing tube (601) and a porous membrane (602). A thermocouple reference wire (603) is disposed within the housing tube 601 in contact with the high-temperature molten salt (HTMS) (604), similar to the HTRE shown in FIG. 1C). The HTRE may include a handle (609), a reference lead wire (610), a housing lead wire (611), and a thermocouple lead wire (612), similar to the HTRE shown in FIG. 5A.

The third electrode wire (619) may be connected to the outside of the housing tube (601) and electrically isolated from the tube using insulator beads (620) and would terminate at the top of the HTRE as an additional lead wire (618). As an alternative to using an insulator bead for isolation, in an embodiment, the portion of the third electrode wire in contact with the housing tube may be coated with an electrical insulator that is chemically and thermally compatible with the reference salt and use-temperature. Non-limiting examples of such insulator materials include non-conductive diamond and boron-nitride.

In an embodiment, the third electrode wire (619) can function as a working electrode, enabling the HTRE to be used as a fully-contained 3-electrode electrochemical cell. The third electrode wire may be Au, Pt, Ni, or any other metal that is chemically compatible at the use-temperature and will provide the desired electrochemical performance characteristics for the desired function. The third electrode wire may also be used in conjunction with the HTRE reference and housing as an electroanalytical sensor to detect the oxygen, water, iron, nickel, chromium, molybdenum, and/or other concentrations in the bulk salt as discussed below.

In an embodiment, the HTRE may contain electronics that can perform self-diagnostic, calibration, and built-in test features. For example, the sensor may be programmed to perform electrical impedance spectroscopy (EIS) or other electroanalytical measurements (current interrupt, conductivity, etc.) to determine the membrane resistance to assess if the membrane has ruptured or is clogged. The sensor would also be able to recalibrate itself using cyclic and square wave voltammetry sweeps and other electroanalytical methods and calibrate itself based on an applicable known redox voltage such as the K⁺/K redox voltage. An integrated working electrode wire, as previously discussed, may be used to perform the electroanalytical methods. Data logging, use-time, estimated remaining life, and communication protocols can also be incorporated into the electronics.

In summary, the disclosed HTRE may provide any or all of the following unique advantages and features:
Laser-cut porous membrane.
Laser-cut porous membrane using thicker substrate that is machined to a thinner membrane only where the pores will be cut.
Stacking multiple layers of the porous-cut membrane to improve stability and to reduce pore size.
Using housing tube that is electrically insulated on the inside for open circuit potential (OCP) measurements and electrochemical impedance spectroscopy (EIS) measurements for HTRE diagnostics.
Using a thermocouple as the reference wire for integrated temperature measurements.
Design kit that enables end users the ability to charge and seal the reference compartment of the HTRE with nuclear-relevant salts of their interest.
Third external electrode that can be used for a fully contained 3-electrode electrochemical cell.
The third external electrode can be utilized with the housing to be an electroanalytical sensor.
Integrated electronics that allow analog to digital conversion, self-diagnostics, calibration, and built-in test features.

### Venturi Flow Tip for a High-Temperature Reference Electrode

The disclosed venturi flow tip, or end cap, may provide some of all of the following features: 1) the venturi flow tip may protect against erosion and corrosion of the HTRE tip and porous membrane, 2) the venturi flow tip may decrease the wicking and diffusion of the reference salt from the reference electrode through the porous membrane, and 3) the venturi flow tip may establish repeatable and gentle hydrodynamic conditions.

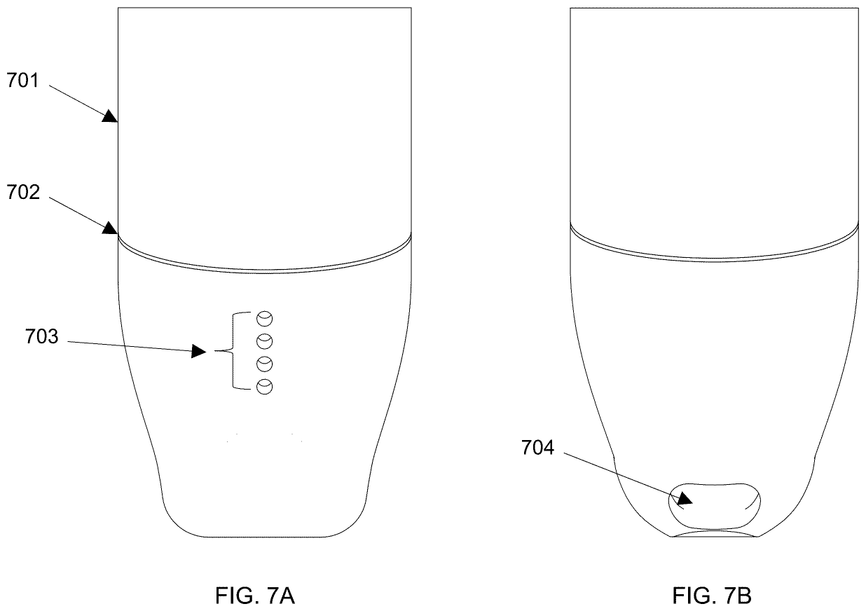

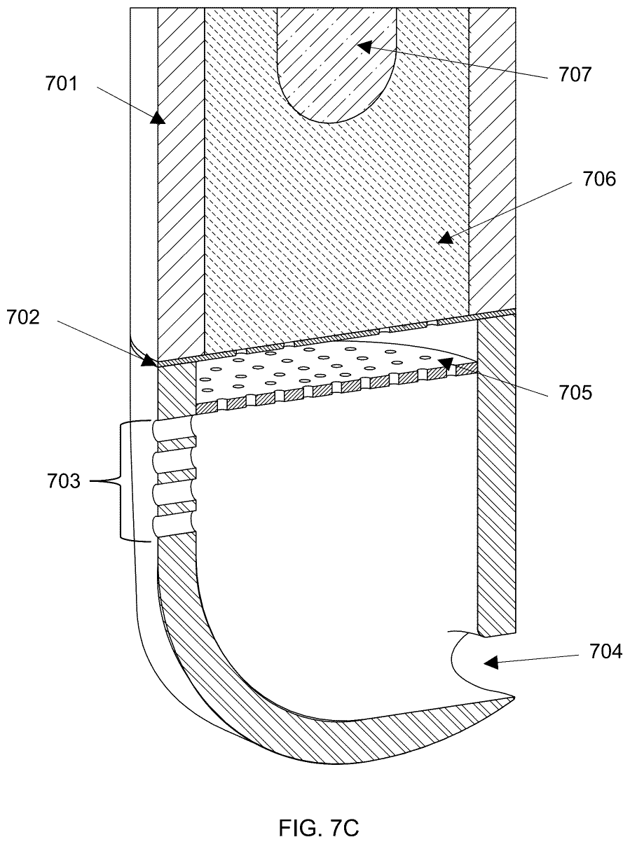

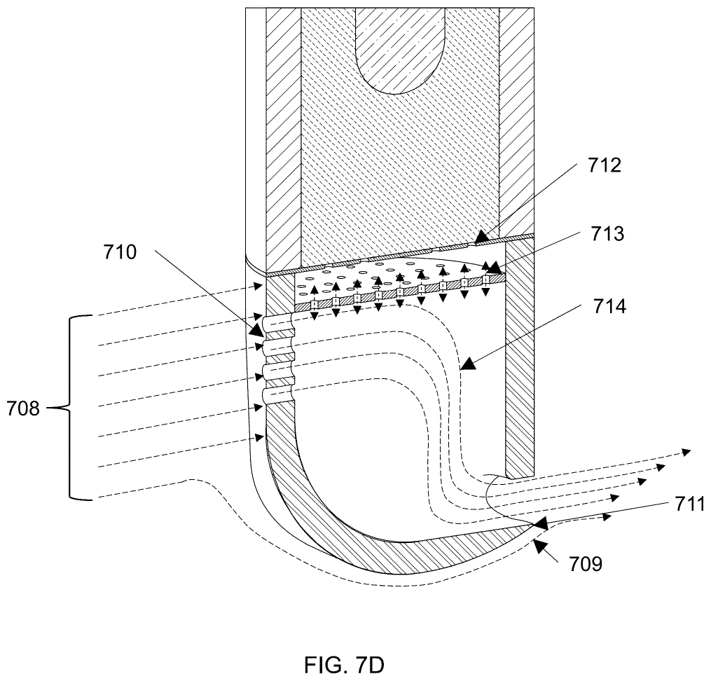

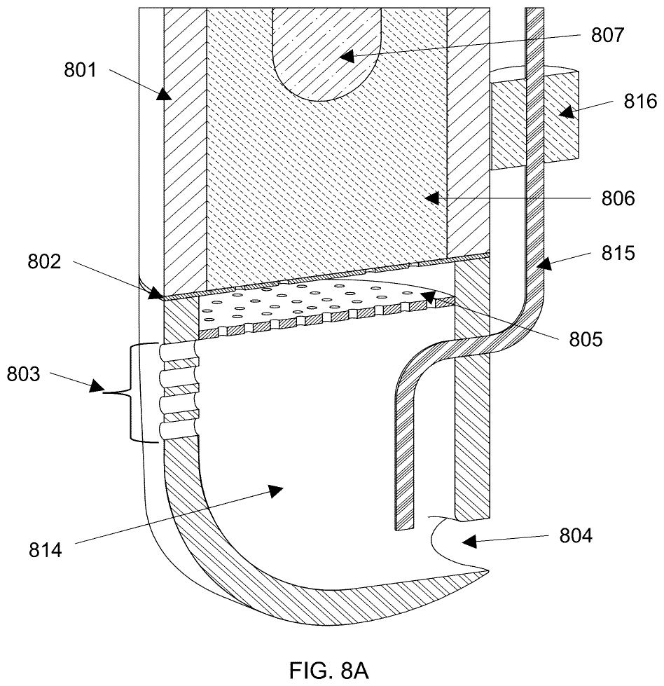

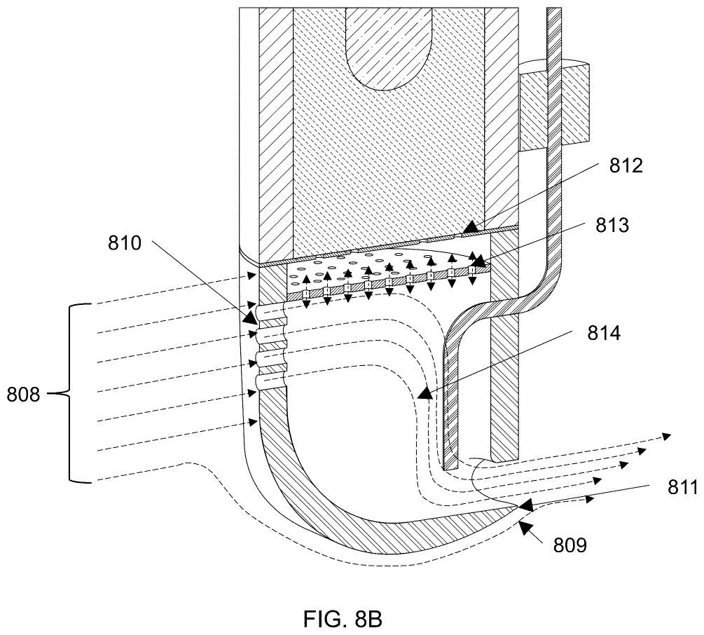

The venturi flow tip can be designed to be placed over any type of reference electrode that may include a porous membrane or frit (702, 802), a housing (701, 801), and a reference wire (707, 807), as shown in FIGS. 7A through 7D and FIGS. 8A and 8B. The flow tip may be screwed on or permanently attached via welding, brazing, or other chemically and thermally compatible method. The flow tip may be removable and replaceable. FIGS. 7A through 7D show an example of one configuration of the venturi flow tip. FIGS. 8A and 8B show an example of the venturi flow tip in a three-electrode HTRE design. The venturi flow tip is designed to use the venturi effect to draw small samples of the test salt into the flow tip's internal chamber (714, 814) for communication with the HTRE's reference salt (706, 806) through the porous membrane (702, 802). The flow tip has an upstream shield with a hole or series of holes (703, 803) at the leading edge of the flow (708, 808) where a stagnation occurs, creating a stagnation (high) pressure (710, 810) as shown in FIG. 7D and FIG. 8B. The downstream end of the cap has a small opening or series of openings (704, 804) where the external flow of the test salt around the cap (709, 809) creates a static (low) pressure (711, 811).

The difference in pressure between the stagnation pressure and the static pressure creates a flow of the test salt into the flow tip chamber (714, 814). The size and/or location of the entrance and exit holes and the shape of the venturi tip may be varied to provide a desired pressure difference. The flow tip design may also provide predictable and controlled hydrodynamic conditions within the flow tip chamber. Design features of the flow tip may be varied and optimized via flow visualization studies, computation fluid dynamics (CFD), or other applicable methods.

The flow tip may contain a baffle (705, 805) just below the porous membrane to further shield the HTRE's porous membrane from the pressure effects and flow effects within the end cap, as shown in FIGS. 7C, 7D, and 8A. In this embodiment the entrained test salt diffuses through the macro-openings (713, 813) of the baffle and then through the micro-openings (712, 812) in the porous membrane.

FIGS. 8A and 8B show the venturi tip in a three-electrode HTRE design. As discussed above, the venturi tip has a structure similar to the venturi tip disclosed in FIGS. 7A-7D.

In the embodiment shown in FIGS. 8A and 8B, the working electrode wire (815) may be inserted into the chamber (814) within the end cap. The working electrode is electrically insulated from the cap and HTRE housing using an insulation bead (816) or other method such as coating the wire. Any electrically insulating method and material that is chemically and thermally compatible may be used, such as beads or wire coatings of boron-nitride, nonconductive diamond, or other similar material.

The disclosed venturi flow tip provides some or all of the following unique features and advantages:

A flow tip for HTREs that uses the venturi effect to draw small samples of the test salt into the cap's internal chamber for ionic exchange with the HTRE's reference salt through the porous membrane.

A baffle within the internal chamber of the flow tip to further shield the HTRE's porous membrane from the pressure effects and flow effects within the flow tip.

A working electrode wire fed into the flow tip chamber enabling the HTRE to function as a fully contained 3-electrode electrochemical cell.

### Engineered Salt Bridge for a High-Temperature Reference Electrode

The disclosed engineered salt bridge provides a means of utilizing commercial off-the-shelf (COTS) reference electrodes (RE) in high-temperature (500 to 1000° C.) molten salts. COTS are readily available from suppliers such as BAS, Pine, Gamry, Koslow, and Ametek in a number of different chemistries and configurations such as Ag/AgCl, saturated calomel electrode (SCE), mercury/mercurous-sulfate, Ni/NiF₂, Ag/AgF₂, etc. This approach enables the use of commercial off-the-shelf reference electrodes in molten salts with less complexity than developing a new high-temperature reference electrode (HTRE).

The engineered salt bridge disclosed herein uses a salt bridge or a Luggin capillary or extension tube and frit that enables ionic communication with the room-temperature COTS RE and the high-temperature test melt and prevents contamination of the reference melt, gel, or solution with the test melt and vice-versa. The reference melt may be any type of molten salt including MgCl₂, KCl, NaCl, AgCl, FLiNaK, FLiBe, or other salt of interest.

Table 1 shows nonlimiting examples of a reference melt, bridge salt, and COTS RE that may be used for specific test melts.

TABLE 1
Example Test Melts, Reference Melts,
Bridge Salts, and COTS REs

| | | | COTS | MP |
|---|---|---|---|---|
| Test Melt | REF Melt | Bridge Salt | REF | (° C.) |
| MgCl₂/KCl/ | MgCl₂/KCl/ | MgCl₂/KCl/ | Ag/AgCl | 396 |
| NaCl | NaCl + AgCl | NaCl + AgCl | | |
| FLiNaK | FLiNaK + AgCl | FLiNaK + AgCl | Ag/AgCl | 454 |

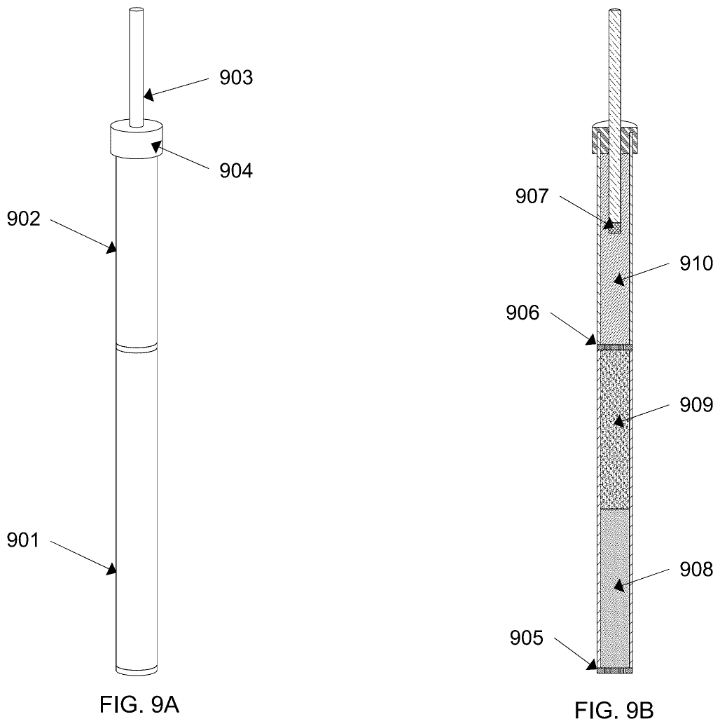

FIGS. 9A and 9B disclose a representation of an engineered salt bridge within the scope of the disclosed invention.

A controlled porosity frit or membrane is sealed to the hot or distal end of the extension tube (905). The frit/porous membrane can be made from any method and material that is chemically and thermally compatible with the test melt. The thermal gradient in the extension tube is controlled by design to keep the cold or proximal end of the COTS RE at room temperature so that COTS REs (903) that are not designed for high-temperature can be used.

The walls of the RE extension tube may be thinned in areas to minimize heat transfer from the high-temperature melt to the COTS RE. Cooling fins may also be attached to the extension tube to promote heat transfer away from the tube.

The COTS RE (903) placed through a seal (904) in the upper end of the RE extension tube (902). Nonlimiting examples of sealing techniques include a compression fitting with PTFE ferrules, a crushed ceramic seal which would minimize heat transfer, a silicone stopper with a retaining ring, or any other chemically and thermally compatible method, which would allow the COTS RE to be replaceable.

The liquid reference melt (908) within the lower extension tube (901) is molten where the RE extension tube is submerged in the test melt and transitions to a solid (909) in the zone outside the test melt where the temperature has cooled to less than the reference salt melting point. The solid portion of the reference salt creates a passive seal the prevents contamination both ways between the COTS RE reference salt and the test melt.

Above the solid reference melt is a bridge salt (910). Nonlimiting examples of the bridge salt include a conductive gel such as KCl dissolved in agar or a gel comprising KCl, glycerol, and cellulose gum. The COTS RE is inserted into the bridge salt and communicates through the frit or porous membrane (907) of the COTS RE.

An optional secondary membrane (906) and compartment may be used between the solid reference melt and conductive gel to minimize contamination or to have a graded concentration. The salt bridge extension is not limited to a straight linear design and may be any shape to accommodate deployment. A nonlimiting example of an alternative shape is an S shape.

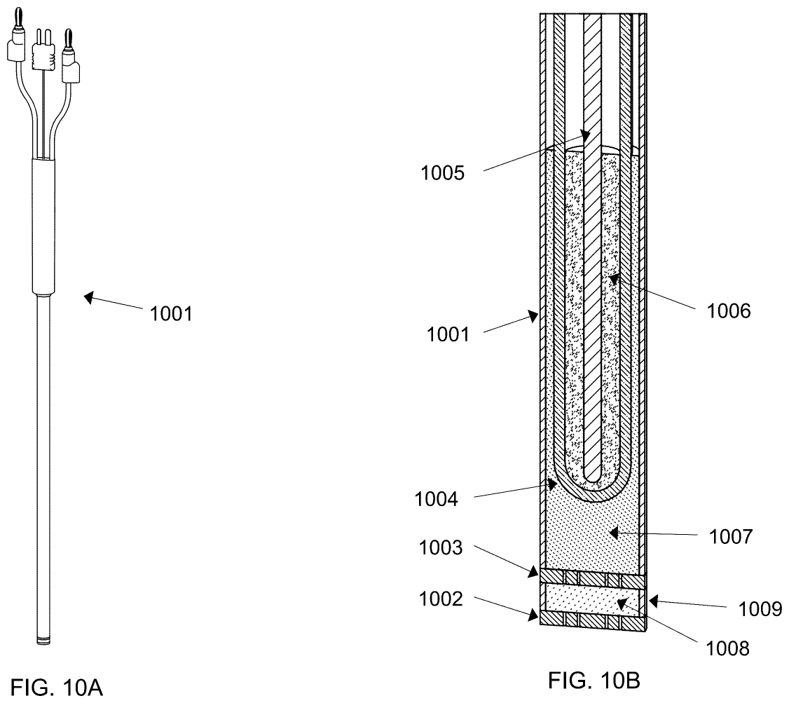

Another embodiment of the invention is disclosed in FIGS. 10A and 10B. FIG. 10A shows the salt bridge design incorporated into a high-temperature reference electrode (HTRE) (1001). FIG. 10B shows a cross-section of the bottom portion of the HTRE. In this embodiment the HTRE contains an outer housing (1001) that is sealed with an ionically communicative membrane (1003). Within the housing/membrane chamber is a bridge salt (1007) that is in ionic communication with an internal membrane (1004) that may, for example, be an ionically communicative ceramic tube such as Mullite, MgO, and BN. Within the internal membrane is the HTRE's molten reference salt (1006) and reference wire (1005). Additionally, the HTRE may contain a second membrane (1002) and tube (1009) that creates an initial chamber that may contain an intermediary salt (1008) to prevent contamination of the bridge salt (1007) to the molten test salt. This embodiment allows for a reference salt to be used that may not be compatible with a metal housing tube (1001) but is compatible with the inner membrane (1004).

The disclosed salt bridge provides some or all of the following unique features and advantages:

A chemically and thermally compatible frit and salt bridge extension tube that allows ionic communication with any COTS RE.

Having both liquid and solid reference salt within the salt bridge, where the solid salt creates a passive seal which prevents contamination both ways between the COTS RE reference salt and the test melt.

1. A high-temperature reference electrode for measuring electrochemical characteristics of a molten salt test composition, comprising:
a metal housing tube comprising an inner surface which defines an inner lumen;
a thermocouple reference wire disposed within the metal housing tube inner lumen and spaced apart from the inner surface, wherein the thermocouple reference wire comprises a reference metal;
a molten reference salt disposed in the metal housing tube in contact with the thermocouple reference wire, wherein the molten reference salt comprises a salt corresponding to the molten salt test composition and a salt of the reference metal; and
a porous membrane comprising pores, wherein the porous membrane is disposed at a bottom end of the metal housing tube.

2. The high-temperature reference electrode of claim 1, wherein the metal housing tube comprises an inner electrical insulating coating at the inner surface.

3. The high-temperature reference electrode of claim 2, wherein the inner electrical insulating coating comprises an insulating material selected from non-conductive diamond and boron-nitride.

4. The high-temperature reference electrode of claim 1, further comprising one or more electrically insulating spacers disposed within the metal housing tube to retain the thermocouple reference wire within the metal housing tube.

5. The high-temperature reference electrode of claim 1, wherein the metal housing tube comprises an outer surface and an outer electrical insulating coating at the outer surface.

6. The high-temperature reference electrode of claim 5, wherein the outer electrical coating comprises an insulating material selected from non-conductive undoped diamond, boron nitride, and silicon carbide.

7. The high-temperature reference electrode of claim 1, wherein the porous membrane comprises two or more plates, wherein each plate comprises a plurality of aligned pores and wherein the plates are offset to cause the pores from one plate to partially overlap the pores of an adjacent plate.

8. The high-temperature reference electrode of claim 1, wherein the porous membrane comprises a porous membrane plate and a membrane support which has a thickness greater than the porous membrane plate.

9. The high-temperature reference electrode of claim 1, wherein the porous membrane comprises a substrate a plurality of pores formed in the substrate, wherein the substrate has a reduced thickness at location where the pores are formed.

10. The high-temperature reference electrode of claim 1, wherein the porous membrane comprises a material chemically compatible with the molten salt test composition selected from mullite, magnesia (MgO), graphite, porous stainless steel, and ion conducting membranes.

11. The high-temperature reference electrode of claim 1, wherein the reference metal comprises Ag.

12. The high-temperature reference electrode of claim 1, wherein the reference metal comprises Ni.

13. The high-temperature reference electrode of claim 1, wherein the metal housing tube comprises a metal which is chemically compatible with the molten salt test composition and the molten reference salt.

14. The high-temperature reference electrode of claim 1, wherein the metal housing tube comprises a metal selected from Ag, Ni, 316-Stainless Steel, and a nickel-chromium-based superalloy.

15. The high-temperature reference electrode of claim 1, wherein the metal housing tube has an inner surface comprising an electrical insulating coating.

16. The high-temperature reference electrode of claim 1, further comprising a stopper seal disposed at a top end of the metal housing tube, wherein the stopper seal comprises a center hole to allow the thermocouple reference wire to pass therethrough.

17. The high-temperature reference electrode of claim 1, further comprising:
a housing lead wire electrically connected to an exterior surface of the metal housing tube; and
a reference lead wire electrically connected to the thermocouple reference wire.

18. The high-temperature reference electrode of claim 1, further comprising an electrode wire connected to an outside surface of the metal housing tube and electrically isolated from the outside surface of the metal housing tube.
